(19) United States
(12) Patent Application Publication
(10) Pub. No.: US 2007/0142348 A1
(43) Pub. Date: Jun. 21, 2007

(54) Microbicidal, prophylactic and therapeutic effect of ctc-96 on papilloma viruses

(75) Inventor: David Gershon, New York, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(73) Assignee: REDOX PHARMACEUTICAL CORPORATION, AUDUBON BIOMEDICAL SCIENCE AND TECHNOLOGY PACK, NEW YORK, NY (US)

(21) Appl. No.: 10/561,323

(22) PCT Filed: Jun. 21, 2004
(86) PCT No.: PCT/US04/19812

§ 371 Date: Dec. 19, 2005

Related U.S. Application Data
(60) Provisional application No. 60/480,206, filed on Jun. 20, 2003.

(51) Int. Cl. A61K 31/555

(52) U.S. Cl. 514/184

(57) ABSTRACT

A method for the therapeutic and prophylactic treatment of subjects infected with the papilloma virus. More specifically, a method of therapeutic treatment of a papilloma virus in a subject by administering an anti-papilloma virus effective amount of CTC-96 to the subject. In addition, a method of prophylactic treatment against a papilloma virus in a subject by administering an anti-papilloma virus effective amount of CTC-96 to the subject to minimize the likelihood of the subject being infected by the papolloma virus.

### BACKGROUND OF THE INVENTION

Sexually transmitted diseases (STDs) are one of our major public health challenges. According to the Center for Disease Control, some STDs, such as syphilis and gonorrhea, have been brought to all time lows. However, strains of HIV resistant to currently used combination therapies are increasingly being identified, and there is a silent and growing epidemic of other STDs that pose equally difficult treatment and prevention challenges. These include genital herpes (HSV-2), Chlamydia and Papilloma. In the United States alone there are over 65 million people with an incurable STD¹. STDs cause serious, life-threatening complications including cancers, infertility, ectopic pregnancy, spontaneous abortions, stillbirth, low birth weight, neurologic damage and death. Whether for a potentially curable or incurable disease a prophylactic approach for the prevention of the spread of STDs would save much human suffering and expense. Papilloma virus (PV), including human papilloma virus (HPV) have both human and veterinary significance, i.e., in cattle, horses, dogs, sheep and birds papilloma viruses in humans' can cause dermal warts, and malignancies, including cervical cancer.

### SUMMARY OF THE INVENTION

We have discovered a method for the therapeutic and prophylactic treatment for subjects infected with Papilloma virus. More particularly, we have discovered that administration of CTC-96 to a subject infected with or who is susceptible to being infected with PV can alleviate the degree of infection or decrease the likelihood that the subject will be infected with PV.

As used herein, the word “therapeutic” means use of the inventive method to treat a subject who has already been infected with Papilloma virus as used herein, the word “prophylactic” means use of the inventive method to protect or decrease the likelihood of a subject who may be exposed to Papilloma virus from being infected with the virus.

Compound CTC-96 has the structure: 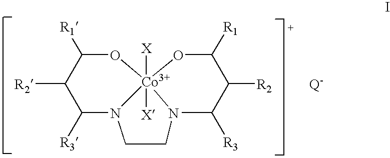
wherein R₁ and R_{1′} are methyl, R₂ and R_{2′} are hydrogen and R₃ and R_{3′} are methyl, and X and X′ are each: 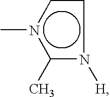
and Q′ is Br⁻.

CTC-96 may be prepared by the method described in the U.S. Pat. No. 5,756,491, the contents of which are hereby incorporated by reference.

Generally, this compound is administered topically in the form of an aqueous solution, but may be administered by other conventional routes.

### BRIEF DESCRIPTION OF THE DRAWINGS

FIG. 1 is a graphical depiction of an evaluation of CTC-96 as a Topical Microbicide

FIG. 2 is a Scatter Plot of Graft Composite Geometric Mean Diameter

### DETAILS OF THE INVENTION

CTC-96™ is one of a new family of chemical entities that are cobalt-containing Schiff base chelates, initially developed as anti-inflammatory agents, capable of scavenging superoxide radicals (U.S. Pat. No. 5,049,557), and subsequently found to have antiviral activity. These agents which do not react with nucleic acid bases are unlike nucleoside analog-type drugs in their mechanism of action. Neither do they act directly as protease inhibitors. Instead, they form stable adducts with the imidazole nitrogen of selected histidines in proteins². They affect viral penetration and cell-to-cell spreading 3. In addition, the drug acts intracellularly by inhibiting viral DNA replication albeit not via direct interaction with the nucleic acid. Because of their different mode of action, These compounds have also shown good efficacy against HSV-1 and HIV viral mutants that are resistant to currently used drugs.

CTC-96™ is taken up by cells and exhibits a distinct ability to unfold specific proteins inside the cells. This can, at least partially, be attributed to the formation of covalent bonds between the Cobalt moiety of CTC-96™ and amino-imidazoles of specific histidines in some proteins. Among these proteins are some Zinc-finger proteins, serine proteases and heme proteins.

Experimental Results:

The following data were obtained without use of a genital formulation. However, CTC-96™ in an appropriate genital formulation also demonstrates a sustained effect and is even more effective than the present data show.

The present invention may be used for both therapeutic and propylactic treatment of various clinical indications results or caused by PV. These include:

Nongenital Skin Warts
Epidermodysplasia Verruciformis
Nonmelanoma Skin Cancer

Infection of the Genital Tract
Anogenital Warts
Cervical Cancer
Nonmalignant and Premalignant Cervical Infection

Anal Cancer

Human Papillomavirus in Other Genital Sites

Penises of male partners of women with cervical dysplasia
Cancers of the vulva, vagina,
Urethral tumors

Human Papillomavirus in the Aerodigestive Tract
RRP (laryngeal papillomas), Recurrent Respiratory Papillomatosis
Cancer of the oral pharynx
Esophageal cancer
Nasal Papillomas
Oral squamous cell Papilloma
Oral condyloma accuminatum
Oral verruca vulgaris
Focal epithelial hyperplasia (FEH)

Conjunctival Papilloma

Evaluation of CTC-96™ in a Human Papillomavirus Type 11-Infected External Human Xenograft-Severe Combined Immunodeficiency (SCID) Mouse Model

The effect of CTC-96™ on the infectivity of HPV-11

The effect of CTC-96™ on the infectivity of HPV-11 was evaluated in the human xenograft SCID mouse model. HPV type 11 is, with HPV-6, one of the two viruses responsible for the majority of anogenital warts in humans.

CTC-96™ in saline was incubated with HPV-11 prior to infection of human neonatal foreskin fragments. The fragments were then grafted onto SCID mice. The animals were monitored weekly. They were weighed at the time of grafting, and every other week during the 12 weeks of the experiment. There was no effect of HPV-11 treatment by CTC-96™ on the weight of the mice. The animals were sacrificed by cervical dislocation 12 weeks after graft implantation. Length, width, and height of the graft were measured and recorded. A composite geometric mean diameter (cGMD) of the grafts was calculated for each mouse. The grafts were then removed and analyzed by histology, immunocytochemistry and RT-PCR. Graft evaluation by immunocytochemistry utilized anti-common Papillomavirus antigen⁴. Explanted grafts were homogenized and total RNA was extracted. HPV-11 viral cDNA was generated by nested RT-PCR.

Effect of CTC-96™ on Graft Size

Comparison of the cGMDs in the treatment groups establishes that regardless of the CTC-96™ concentration there was a small but significant effect on the infectivity of HPV-11 when compared to the control (no CTC-96™) (Table 1 ).

TABLE 1
Composite Geometric Mean Diameters (cGMD) of the Grafts (mm)

| Treatment | N | Mean ± SD | Median |
|---|---|---|---|
| CTC-96 ™ 0% | 9 | 2.58 ± 0.808 | 2.59 |
| CTC-96 ™ 0.05% | 8 | 2.02 ± 0.179 | 2.01 |
| CTC-96 ™ 0.2% | 6 | 1.86 ± 0.196 | 1.88 |
| CTC-96 ™ 1% | 9 | 1.95 ± 0.139 | 1.94 |

Effect of CTC-96™ and HPV-11 Treatment on Graft Histology

Table 2 shows the results of the histologic examination of the grafts for the presence of HPV. Presence of HPV was defined by the presence of two out of three of the following features: acanthosis (an increase in the thickness of the stratum spinosum of the epidermis) koilocytosis (perinuclear vacuolation), or parakeratosis (persistence of the nuclei in the cells of the stratum corneum of the epidermis). As the CTC-96™ concentration increases the percentages of grafts containing HPV decreases, with none positive at the two higher concentrations. At these two highest CTC-96™ concentrations (0.2% and 1%) the vast majority of the grafts displayed a fibrous tissue or a foreign body reaction and were thus classified as non-interpretable. Two explanations are possible for the high number of foreign body reactions present in the grafts infected with the HPV-11 treated with the two highest concentrations of CTC-96™. The first is that CTC-96™ completely inactivated HPV-11. Hence the grafts were not infected, and as a consequence were more likely to be eliminated by a foreign body reaction. The alternate explanation is that CTC-96™ was toxic to the graft itself. This is unlikely since CTC-96™ has been used in vitro and in vivo in intravaginal preparations without associated toxicities at the concentrations used, and at the lowest concentration of CTC-96™ (0.05%), the number of grafts with a foreign body reaction was not different than that in the control group, thus toxicity would be unlikely to account for the lower number of HPV positive grafts (P=0.0 15; by Fisher exact test). Since CTC-96™ has a clear virucidal action at the lowest concentration (see Table 4), it is reasonable to assume that this virucidal action is present at higher concentrations, independent of the presence of a cytotoxic effect.

Table 2 presents the results when grafts exhibiting a foreign body reaction or fibrous tissue were counted as negative for HPV. The conclusion that CTC-96™ has virucidal activity against HPV-11 remains identical to that of the previous analysis, but with more statistical significance because of the greater number of observations.

TABLE 2
Histology CTC-96 ™ and HPY-11 Treated on Grafts *

| | | | |
|---|---|---|---|
| Treatment | Negative | Positive | Totals |

| | | | | |
|---|---|---|---|---|
| CTC-96 ™ 0% | 6 | 11 | (64.7%) | 17 |
| CTC-96 ™ 0.05% | 11 | 4 | (26.7%) | 15 |
| CTC-96 ™ 0.2% | 12 | 0 | (0%) | 12 |
| CTC-96 ™ 1% | 16 | 0 | (0%) | 16 |

* In this analysis, observations counted as negative include grafts that were read as negative for HPV as well as grafts that displayed a foreign body reaction or some fibrous tissue

In Table 3 grafts exhibiting foreign body reaction or fibrous tissue are considered negative for HPV. The conclusion that CTC-96™ has virucidal activity against HPV-1 is unchanged.

TABLE 3
Immunocytochemistry of CTC-96 ™ and HPV-11 Treated on Grafts *

| | | | |
|---|---|---|---|
| Treatment | Negative | Positive | Totals |

| | | | | |
|---|---|---|---|---|
| CTC-96 ™ 0% | 10 | 7 | (41.2%) | 17 |
| CTC-96 ™ 0.05% | 14 | 2 | (12.5%) | 16 |
| CTC-96 ™ 0.2% | 12 | 0 | (0%) | 12 |
| CTC-96 ™ 1% | 17 | 0 | (0%) | 17 |

* Histology of the grafts was reviewed for the presence of HPV as measured by immunocytochemistry using anti-common Papillomavirus antigen⁴. In this analysis, observations counted as negative include grafts that were read as negative for HPV as well as grafts that displayed a foreign body reaction or some fibrous tissue

Effect of CTC-96™ on Graft HPV RT-PCR

Table 4 presents the results and analysis of the HPV RT-PCR, which was performed by extracting total RNA from homogenized, explanted grafts and generating HPV-11 viral cDNA by nested RT-PCR. Using this assay, there is a strong and clear dose-response effect. Only with the highest dose of CTC-96™ is there complete obliteration of viral transcriptional activity. In contrast with the histology and immunocytochemistry results where the two highest doses of CTC-96™ did not show evidence of the presence of HPV, with the RT-PCR, HPV cDNA was detected in two grafts from the CTC-96™ 0.2% group.

TABLE 4
RT-PCR of CTC-96 ™ and HPV-11 Treated Grafts

| | HPV-11 | HPV-11 | | Non- | |
|---|---|---|---|---|---|
| Treatment | Negative | Positive | | Interpretable* | Totals |
| CTC-96 ™ 0% | 4 | 13 | (76.5%) | 0 | 17 |
| CTC-96 ™ 0.05% | 7 | 9 | (56.2%) | 0 | 16 |
| CTC-96 ™ | 5 | 2 | (28.6%) | 5 | 12 |
| CTC-96 ™ 1% | 15 | 0 | (0%) | 2 | 17 |

* These samples were not interpretable because both the HPV and P-actin messages were not detected.

The RT-PCR results support a virucidal rather than a toxic effect of CTC-96™.

### CONCLUSIONS

The treatment of HPV-11 with CTC-96™ had an inhibitory effect on the infectivity of the virus as measured by graft size in the human xenograft SCID mouse model with the three tested concentrations of CTC-96™ leading to a drastic inhibitory effect on graft size when compared to the control group. Analysis of the presence of HPV by histology, immunocytochemistry, and RT-PCR, demonstrated a clear dose-response effect. The virucidal effect of the lowest concentrations of CTC-96™ was only partial while the highest concentration of CTC-96™ (1%) appeared completely virucidal. CTC-96™ did not exert its action by direct toxicity on the grafts. As analyzed by RT-PCR, the vast majority of the grafts were viable at the end of the experiment despite lacking evidence of HPV transcription. CTC-96™ had no effect on the animals' mortality or weight gain. CTC-96™ did not stimulate viral or cellular replication.

Evaluation of CTC-96™ Therapeutic Activity on Human Condylomas Induced by HPV-11 in the HPV-11-Infected External Human SCID Mouse Model

Two different dose levels of CTC-96™ (1%, 0.1%) in an ointment formulation and the vehicle alone were evaluated. SCID mice were grafted on each side of the dorsum with an HPV-11-infected foreskin fragment. The HPV-11-infected grafts were left to grow for 6 weeks before treatment was started and continued for 6 weeks. During the treatment phase, the drug was administered thrice a week, directly on the graft. Graft length, width, and height were measured every two weeks during the treatment phase. At the end of the study, the animals were sacrificed. The grafts were measured, recovered, and processed for histology. HPV infection in tissue sections is defined by the presence of two out of three of the following features: acanthosis, koilocytosis, or parakeratosis.

Effect of CTC-96™ on Graft Size

The Graft Size Growth (GSG) index was our primary endpoint in this evaluation of CTC-96™. Table 5 provides the summary statistics of this measurement. There is a gradual increase in the mean GSG with higher CTC-96™ concentration, which would suggest, that CTC-96™ stimulates the growth of Papillomavirus-infected tissue. However, there is also a marked increase in the variance of the GSG in the high dose CTC-96™ group.

TABLE 5
Graft Size Growth (%)

| Treatment | N | Means ± SD | Median |
|---|---|---|---|
| CTC-96 ™ 0% | 0 | 57.50 ± 48.59 | 50.65 |
| CTC-96 ™ 0.1% | 14 | 64.53 ± 41.92 | 60.51 |
| CTC-96 ™ 1% | 16 | 91.39 ± 127.84 | 52.03 |

In our analysis, to measure graft size growth we used a composite index that summarizes for each mouse the growth of the two grafts borne by the animal, each from a different foreskin donor. If instead we look at the growth of each individual graft, we can see that the means, medians, or variances of the graft size growth are more alike among the three treatment groups as shown in FIG. 1. The ANOVA fails to show a treatment effect on the growth of the individual grafts.

CTC-96™ has been found to be virucidal against Human Papilloma Virus (HPV) Type 11 with no detectable effect on the growth of HPV-11-induced papillomas CTC-96™ neither decreased nor increased the growth of HPV- 11-infected human papillomas. Given the virucidal activity of CTC-96™, a series of experiments was performed to evaluate the prophylactic activity of the compound under conditions resembling that of the use of a topical microbicide. When using a topical microbicide the target organ is first exposed to the microbicide before exposure to the virus. Various concentrations of CTC-96™ were evaluated in a model of HPV-11-infected human xenograft in the SCID mouse. The human grafts were exposed to CTC-96™ for 1 hour prior to exposure to HPV-11 and engraftment. Because CTC-96™ is virucidal, it was washed off the foreskin fragments before exposure to the virus. The HPV-11-infected grafts were allowed to grow for 12 weeks. After 12 weeks, the animals were sacrificed and the grafts recovered, measured and processed. Graft size, expressed as the composite geometric mean diameter of the two grafts borne by the animal, was the primary endpoint. Histology of the grafts was examined for the presence of HPV. Grafts were also processed for detection of HPV-11 mRNAs transcripts by reverse transcriptase-polymerase chain reaction (RT-PCR).

Results are in agreement with a dramatic and highly statistically significant microbicidal effect of CTC-96™ as shown in FIG. 2. Graft size in the control group was significantly larger than that in each of the groups in which CTC-96™ was used. However, the differences were not statistically significant between the groups in which the foreskin fragments were treated with CTC-96™.

Table 6 summarizes the histology results for the presence of HPV in the grafts. As the CTC-96™ concentration increased, the number of grafts displaying either fibrous tissue or a foreign body reaction also increased. Grafts that are not infected by HPV proliferate poorly and tend to be eliminated by the mouse's host defenses as a foreign body. The residual fibrous tissue is a scar. In order to analyze the results properly, grafts that were truly negative (graft showing a healthy human epithelium) as well as those showing a foreign body reaction or fibrous tissue were counted as negative for HPV. These results also suggest that CTC-96™ has a microbicidal effect on HPV-11.

TABLE 6
Histology Results for the Presence of HPV

| | Histology | | | |
|---|---|---|---|---|
| Treatment Group | | Negative | Positive | Total |

| | | | | | |
|---|---|---|---|---|---|
| CTC-96 ™ 0% | 3 | 10.5% | 17 | 89.5% | 19 |
| CTC-96 ™ 0.05% | 7 | 30% | 16 | 73% | 23 |
| CTC-96 ™ 0.2% | 14 | 67% | 7 | 33% | 21 |
| CTC-96 ™ 1% | 22 | 100% | 0 | 0% | 22 |

Table 7 summarizes the immunocytochemistry results for the presence of HPV capsid antigen in the grafts. As the concentration of CTC-96™ increases, the number of grafts displaying either fibrous tissue or a foreign body reaction also increases. Therefore, the table was constructed such that the grafts demonstrating fibrous tissue or a foreign body reaction were counted as negative. Again, a strong microbicidal effect of CTC-96™ is observed.

TABLE 7
Modified immunocytochemistry Results for the Presence of HPV

| | Immunocytochemistry | | |
|---|---|---|---|
| Treatment Group | Negative | Positive | Total |

| | | | | | |
|---|---|---|---|---|---|
| CTC-96 ™ 0% | 3 | 16% | 16 | 84% | 19 |
| CTC-96 ™ 0.05% | 11 | 48% | 12 | 52% | 23 |
| CTC-96 ™ 0.2% | 15 | 71% | 6 | 29% | 21 |
| CTC-96 ™ 1% | 22 | 100% | 0 | 0% | 22 |

Table 8 summarizes the results of the analysis of the grafts for HPV-11 expression. These results indicate that 22% of the grafts still showed viral expression with the highest concentration of CTC-96™. This demonstrates that viral infection is not necessarily accompanied by tissue proliferation. Some of the grafts that demonstrated the presence of fibrous tissue or foreign body reaction also contained HPV mRNA.

TABLE 8
RT-PCR Results for the Presence of HPV

| | RT-PCR | | |
|---|---|---|---|
| Treatment Group | Negative | Positive | Total |

| | | | | | |
|---|---|---|---|---|---|
| CTC-96 ™ 0% | 5 | 22% | 18 | 78% | 23 |
| CTC-96 ™ 0.05% | 9 | 39% | 14 | 61% | 23 |
| CTC-96 ™ 0.2% | 17 | 81% | 4 | 19% | 21 |
| CTC-96 ™ 1% | 14 | 78% | 4 | 22% | 18 |

In conclusion, CTC-96™ was shown to have a strong effect on HPV-11 when used under conditions simulating the natural infection. Although CTC-96™ did not completely block infection, even at the highest concentration (1%), the clinical markers of infection were absent (graft proliferation, signs of HPV infection by histology and immunocytochemistry).

Inactivation of Bovine Papillomavirus Type 1 (BPV-1) by CTC-96™

Experiments were performed to determine if CTC-96™ can inactivate the ability of bovine papillomavirus type 1 (BPV-1) to morphologically transform C127 mouse epithelial cells in culture. Cell-free stocks of BPV-1 were treated with CTC-96™ or placebo. In order to detect the transforming ability of BPV-1, sub-confluent cultures of C127 mouse cells are infected with a standardized inoculate of BPV-1. For the positive controls, stock virus was added to the culture medium present on the cells. The presence of morphologically transformed foci was counted after 2 weeks and then again at 3 weeks. Controls included non-infected cells (with or without drug) and untreated BPV-1.

Bovine Papillomavirus Type 1 (BPV-1) was mixed with CTC-96™, incubated for 10 minutes at 37° C. and then added to the cells. It is clear that CTC-96™ can inhibit the appearance of bovine Papillomavirus type 1 (BPV-1) induced transformation of C127 mouse epithelial cells in culture (Table 9).

TABLE 9
CTC-96 ™ Inhibition of Bovine Papillomavirus Type 1 (BPV-1)
Transformation of C127 Mouse Epithelial Cells in Culture: Exposure
of Virus to Drug Prior to Infection.

| | | |
|---|---|---|
| | | Number of |
| | CTC-96 ™ | Transformed |
| Treatment | (mg/ml) | Foci at 3 weeks |

| | | | |
|---|---|---|---|
| PBS alone | — | 0 | 0 |
| BPV-1 alone | | 96 | 105 |
| CTC-96 ™ | 0.1 | 68 | 80 |
| CTC-96 ™ | 0.2 | 80 | 82 |
| CTC-96 ™ | 0.5 | 25 | 40 |

To further evaluate the time course of interaction between CTC-96™ and Papillomavirus the following experiment was performed. Virus was put on C127 mouse epithelial cells in culture at time 0 and incubated for 5 hours. Medium was then removed and replaced with medium plus drug. Cells were then re-fed every 2 days and the experiments counted on day 12. The results shown in Table 10 suggest an effect of the compound on the virus in the infected cells, and not on extracellular virus.

TABLE 10
CTC-96 ™ Inhibition of Bovine Papillomavirus Type 1 (BPV-1)
Transformation of C127 Mouse Epithelial Cells in Culture: Exposure
of Cells Pretreated with Virus to Drug.

| | | Number of Transformed | |
|---|---|---|---|
| | CTC-96 ™ | Foci | |
| Treatment | (μg/ml) | Exp. 1 | Exp. 2 |

| | | | | | |
|---|---|---|---|---|---|
| PBS alone | — | 0 | 0 | 0 | 0 |
| BPV-1 alone | — | 81 | 72 | 41 | 43 |
| Placebo | — | 77 | 58 | 20 | 21 |
| CTC-96 ™ | 5 | 3 | 2 | 3 | 3 |

To further clarify the dynamics of CTC-96™ Papillomavirus interaction an experiment was performed to combine exposure of virus to the drug prior to cellular infection and exposure of infected cells to the drug. For this purpose BPV-1 was incubated with either CTC-96™ or placebo, diluted and then added to C127 Mouse Epithelial Cells in culture with the addition of CTC-96™ or placebo (Table 11). Addition of 10 μg/ml or 20 μg/ml CTC-96™ to the cell cultures was toxic to the cells. Addition of 5 μg/ml CTC-96™ effectively suppressed all focus formation. Pre-treatment with 50 μg/ml CTC-96™ but not placebo halved the foci number in the normal medium post-treatment control. Thus a small microbicidal effect is probable even at these low concentrations of CTC-96™. The main activity at these concentrations, however, is post-infection. However, pretreatment of the virus (microbicidal effect) also caused 50-60% inhibition of the virus.

TABLE 11
CTC-96 ™ Inhibition of Bovine Papillomavirus Type 1 (BPV-1)
Transformation of C127 Mouse Epithelial Cells in Culture:
Pretreatment and Post-treatment combined

| | | | Number of Transformed Foci | | | |
|---|---|---|---|---|---|---|
| Pre-treatment | | Post-treatment | Experiment 3-1 | | Experiment 3-2 | |
| Mock inoculum | | Normal medium | 0 | 0 | 0 | 0 |
| BPV | | Normal medium | 163 | 156 | 183 | 148 |
| Placebo | BPV | normal medium | 175 | 117 | 155 | 149 |
| Placebo | BPV | placebo | 150 | 130 | 133 | 122 |
| Placebo | BPV | 5 μg/ml CTC-96 | 0 | 0 | 0 | 0 |
| 50 μg/ml | BPV | normal medium | 71 | 46 | 46 | 41 |
| CTC-96 | | | | | | |
| 50 μg/ml | BPV | placebo | 65 | 61 | 39 | 62 |
| CTC-96 | | | | | | |
| 50 μg/ml | BPV | μg/ml CTC-96 | 0 | 0 | 0 | 0 |
| CTC-96 | | | | | | |

What is claimed is:

1. A method of therapeutic treatment of a disease caused by papilloma virus in a subject comprising administering an anti-papilloma virus disease effective amount of CTC-96 to the subject.

2. The method of claim 1 wherein the subject is human.

3. The method of claim 1 wherein the subject is a mammal.

4. The method of claim 1 wherein the subject is an animal.

5. The method of claim 4 wherein the subject is a cow, steer, horse, dog sheep or bird.

6. The method of claim 1 wherein the disease is selected from the group consisting of nongenital skin arts, Infection of the Genital Tract, Anal Cancer, Human Papillomavirus in Other Genital Sites, Penises of male partners of women with cervical dysplasia, and Conjunctival Papilloma.

7. The method of claim 6 wherein the disease is selected from the group consisting of Epidermodysplasia Verruciformis, Nonmelanoma Skin Cancer, Anogenital Warts, Cervical Cancer, Nonmalignant and Premalignant Cervical Infection, Cancers of the vulva, vagina, Urethral tumors, RRP (laryngeal papillomas), Recurrent Respiratory Papillomatosis, Cancer of the oral pharynx, Esophageal cancer, Nasal Papillomas, Oral squamous cell Papilloma, Oral condyloma accuminatum, Oral verruca vulgaris, .and Focal epithelial hyperplasia (FEH)

8. A method for the complete or partial avoidance of a disease caused by a papilloma virus in a subject comprising administering anti papilloma virus prophylactic effective amount of CTC-96 to a subject to minimize or avoid the subject's being infected by a papilloma virus.

9. The method of claim 8 wherein the subject is human.

10. The method of claim 8 wherein the subject is a mammal.

11. The method of claim 8 wherein the subject is an animal.

12. The method of claim 11 wherein the subject is a cow, steer, horse, dog sheep or bird.

13. The method of claim 8 wherein the disease is selected from the group consisting of nongenital skin arts, Infection of the Genital Tract, Anal Cancer, Human Papillomavirus in Other Genital Sites, Penises of male partners of women with cervical dysplasia, and Conjunctival Papilloma.

14. The method of claim 13 wherein the disease is selected from the group consisting of Epidermodysplasia Verruciformis, Nonmelanoma Skin Cancer, Anogenital Warts, Cervical Cancer, Nonmalignant and Premalignant Cervical Infection, Cancers of the vulva, vagina, Urethral tumors, RRP (laryngeal papillomas), Recurrent Respiratory Papillomatosis, Cancer of the oral pharynx, Esophageal cancer, Nasal Papillomas, Oral squamous cell Papilloma, Oral condyloma accuminatum, Oral verruca vulgaris, and Focal epithelial hyperplasia (FEH)
